(19) United States
(12) Patent Application Publication
(10) Pub. No.: US 2006/0089521 A1
(43) Pub. Date: Apr. 27, 2006

(54) Rotor driven linear flow blood pump

(76) Inventor: Sheldon S. L. Chang, Port Jefferson, NY (US)

Correspondence Address: Sheldon S. L. Chang, 5 Seaside Drive, P.O. Box 273, Port Jefferson, NY, 11777-0273, US

(21) Appl. No.: 10/969,676

(22) Filed: Oct. 21, 2004

(51) Int. Cl. A61N 1/362

(52) U.S. Cl. 600/16

(57) ABSTRACT

A rotor driven linear flow blood pump (LFBP) which completely separates the driving motor from the pumped blood is used as a vascular assist device (VAD). Without the possible hazard of blood contamination, a brushless d. c. motor (BLDC) is ideal to drive and to control the LFBP. Thus we have the best of two worlds: For patient mobility, d. c. batteries are the best as a VAD energy source, and LFBP provides the most means at a physician's disposal for curing his patient with a severely damaged heart. A key to success in making the above possible is a new concept of surface affinity. Complete blood containment is made possible by using a material with zero surface affinity with blood as the bearing material throughout.

### BACK GROUND OF THE INVENTION

Among various types vascular assist devices (VAD), the linear flow blood pump (U.S. Pat. No. 6,361,292 B1) has the sole advantage of moving blood forward without using valves. It also has the means for controlling the pressure and flow volume separately. These advantages and means can be utilized to restore some damaged heart to good health. Thus an linear flow blood pump can be used as the foundation of an enhanced vascular assist device (EVAD), which not only can delay the need of a heart transplantation, but also may eliminate the need of heart transplantation altogether by restoring the native heart to good health.

The pumping motion of a linear flow blood pump is carried out through the relative motion of two elements. An outer element, and an inner element. In the device described in U.S. Pat. No. 6,361,292 B1, the outer element is rotated by an electrical motor, with the inner element following the outer element movement. In the present application, the inner element is driven with the outer element following the inner element movement. Both versions are worth developing because of their complementary advantages.

### BRIEF SUMMARY OF THE INVENTION

The invention herein is a ventricular assistive device based on a progressive cavity pump having an outer element, an inner element with a drive shaft, and a sealing bearing at the driving end of the drive shaft. In addition to its normal functions, the sealing bearing meets the following objectives:
(a) Keeping the pumped blood from leaking out.
(b) Allowing blood to flow freely without accumulation.

The above are accomplished by means of:
(i) Using more than one sealing section in the sealing bearing.
(ii) Using a bearing material which has no surface affinity with blood for each and every sealing section.
(iii) Providing a feedback connection from the sealing bearing to the pump inlet.

The main advantage of the present invention is its complete separation of the drive motor from blood, and thereby a Brushless d. c. motor (BLDC) can be used to drive and to control the pump operations. Since either a d. c. battery or a d. c. storage battery is used as the power source, direct use of a BLDC represents substantial cost and weight savings, as well as simplicity and reliability.

### BRIEF DESCRIPTION OF THE DRAWINGS

FIG. 1 Rotor Driven Linear Flow Blood Pump

FIG. 2 Sealing Bearing and Bearing Sections

### DETAILED DESCRIPTION OF THE INVENTION

In the detailed description, we follow the following sequence:

1. Mathematical Description

1.1 Normal Operation of a Moineau Pump

1.2 Preferred Embodiment of the Invention

2. The concept of Surface Affinity

3. Detailed Discussion of the Figures.

1. Mathematical Description

1.1 Normal Operation of a Moineau Pump

In normal pump operation, the stator is stationary, and the rotor rotates. Since at every axial position, z, the rotor is circular, we can describe the rotor movement by its center r, where r is a complex value r=x+jy:
r=Ee^{j(θ-k)}+Ee^{-jθ} (1)

In (1), E is the pump eccentricity, θ is a time variable, and k is related to the rotor pitch pᵣ:

$$\begin{matrix}θ = ( \frac{n}{2 π} ) t & ( 2 ) \\ k = ( \frac{2 π}{{p}_{r}} ) & ( 3 )\end{matrix}$$

where n is the number of rotor revolutions per second, and pᵣ is the rotor pitch length: kz increases by 2π, with each increase of z by pᵣ.

In (1), the first term on the right hand side represents rotor rotations, and the second term represents the rotor movement as a whole, or mutation. The direction of mutation is opposite to that of rotation, and is a result of rotor stator interaction. The stator contour is generated to fit into the rotor motion. Equation (1) can be rewritten as:
r=Ee^{-jkz/2}(e^{j(θ-kz/2)}+e^{-j(θ-kz/2)})=2Ed^{-jkz/2} cos(θ−kz/2) (4)

Equation (4) has the following significances: (a) It represents the way Moineau pump stator is built. At each value of z, the rotor movement defines an envelope with one semi-circle of diameter D at each end, where D is the rotor cross-sectional diameter, with two connecting lines of 4E on each side. (b) The longitudinal direction of the stator rotates with z. As kz/2 increases by 27π, the stator returns to its original direction, ie:

$$Δ ( kz / 2 ) = 2 π \\ {p}_{s} = Δ z = \frac{4 π}{k} = 2 {p}_{r} \qquad ( 5 )$$

Thus the stator pitch pₛ is twice the rotor pitch. In common usage among pump engineers, pₛ is also referred to as pump pitch, or P. Thus we shall adopt this usage from now on. (c) At any value of z, the stator is separated into two areas by the rotor. As z varies two closed pockets are formed. The terminal values of z for one pocket is given by

$$θ - \frac{kz}{2} = \pm \frac{π}{2} \qquad ( 6 )$$

Solving z from (6) gives

$$z = \frac{2}{k} ( θ \pm \frac{π}{2} ) \qquad ( 7 )$$

Equation (7) gives the two terminal values of z forming a single closed pocket:

$$\begin{matrix}{z}_{-} = \frac{2}{k} ( θ - \frac{π}{2} ) & ( 8 ) \\ {z}_{+} = \frac{2}{k} ( θ - \frac{π}{2} ) & ( 9 )\end{matrix}$$

As the two terminals move with θ, the whole pocket moves with θ in the direction of increasing z. The central point of the pocket is given by

$${z}_{c} = \frac{1}{2} ( {z}_{+} + {z}_{-} ) = \frac{2}{k} θ \qquad ( 10 )$$

Equation (10) can be rewritten as

$${z}_{c} = \frac{2}{k} \cdot 2 π nt = Pnt \qquad ( 11 )$$

From (11), we see that the pocket moves forward by one stator pitch P with each revolution. The pump output volume is given by
V=nP(statorarea−rotorarea)=nP(eDE)=4π(DEP) (12)

Equation (12) is the well known Moineau pump output equation. Since z_{c} is the central point of a closed pump pocket, we can use z_{c} to represent the pump pocket. Substituting z_{c} for z in (4) gives the rotational motion of z_{c}
r_{c}=2Ee^{-jθ} (13)
1.2 Preferred Embodiment of the Invention

If we rotate the entire system including both the rotor and the stator by an angle e^{jθ}, the output volume remain the same as (12). However the actual rotor speed N is
N=n+n=2n (14)

Expressing (12) in terms of N gives
V=2NDEP (15)

To determine the pump pocket motion, we obtain from (13):
e^{jθ}r_{c}=2E (16)

Thus the stator rotates at the speed of n which is the same as N/2 revolutions per second about the r=0 axis. We shall refer to the r=0 axis as the central axis. Equation (16) shows that the pump pocket forward movement does not rotate and its forward motion is given by (11). The pump pocket's rotational motion is obtained by substituting z_{c} for z in (13). Since θ−kz_{c}/2=0, (13) yields
e^{jθ}r_{c}=2E (17)

Equation (17) means that in the rotational system there is no rotational movement of the pump pockets. Combining ( 11) and (17), we see that in the rotational system, the pump pockets move straight forward at speed Pn.

2. The Concept of Surface Affinity

The concept of surface affinity is very pertinent to the present invention, but it may very well be a novel concept. For instance, if we drop a drop of blood on a glass surface or a steel surface, the drop of blood would spread and stick to the surface. If we wish to get the blood off, we must have the surface washed. On the other hand, if we drop a drop of blood on a Teflon surface, the drop of blood would stay together as a drop. If we tilt he Teflon surface somewhat, the blood would roll off, with no trace left on the Teflon surface. The reason is that there is no surface affinity between blood and Teflon. But there is definitely some surface affinity between blood and most other materials. Now that the steel shaft definitely has some surface affinity. If the bearing material also has surface affinity, blood would spread on both the bearing surface and shaft surface, and seep through readily. If Teflon is used as the bearing material, blood cannot spread on the Teflon surface. With normal clearance between shaft and bearing, blood cannot leak through as a drop either. Then blood may not seep through at all, or at least not as readily. It is with this idea in mind. I looked through various references including CRC's Handbook of Chemistry and Physics, and did not find anything on the concept of surface affinity. Then I felt that maybe I should explain this concept in detail here.

3. Detailed Description of the Figures

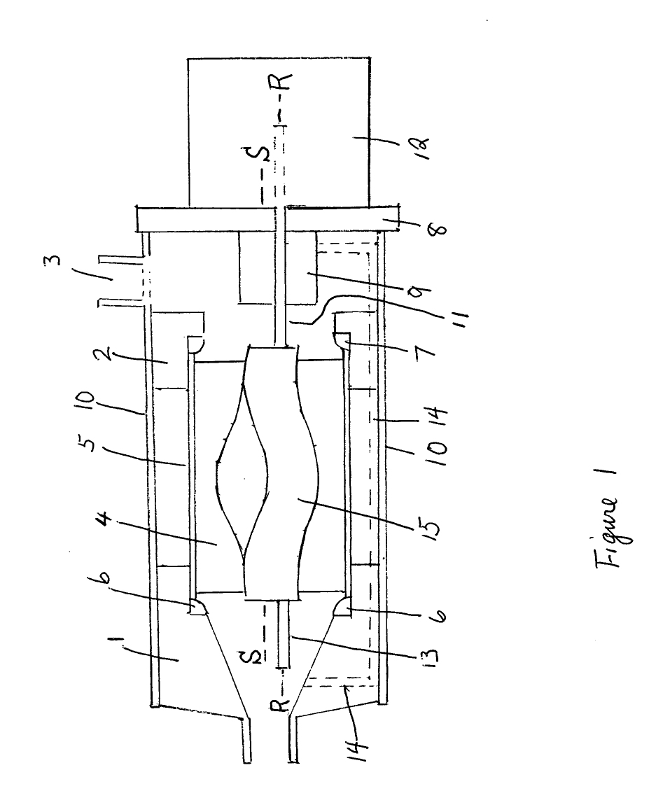

FIG. 1 is an illustration of a preferred embodiment of the present invention. The central or stator rotational system axis is denoted as SS. The rotor rotational system axis is denoted as RR. The two axis are parallel and spaced at a distance E apart. In FIG. 1, I is the inlet bearing including the inlet port; 2 is the outlet bearing; 3 is the outlet port; 4 is the pump stator with stator shell 5; 6 is an extension of 5 at the inlet end; 7 is an extension of 5 at the outlet end; We note that 6 and 7 increase very substantially the vertical bearing area and thereby improving the bearing life 10 is an outside tubing holding within all the other components of the device including the pumped blood; 8 is an end cover plate at the outlet end; 9 is a sealing bearing for rotor shaft 11 at the outlet end; the rotor shaft 11 connects to the drive motor 12 and is powered by 12. The rotor shaft 13 at the inlet end can be either free or supported by a spider-connected bearing which is not shown in FIG. 1. The components 1, 2, 5, 6, 7, 8, are rotarily symmetrical about the SS axis, with exceptions at the opening for 3, through hole for 11 at 9 and 8, and feedback path 14. The sealing bearing 9 is rotarily symmetrical about the RR axis.

There are one stationary system and two rotational systems in FIG. 1. The stationary system includes 1, 2, 3, 8, 9 10, and 14. The stator rotational system includes 4, 5, 6, 7, and rotates about the SS axis. The rotor rotational system includes 11, 13, and 15 and rotates about the RR axis.

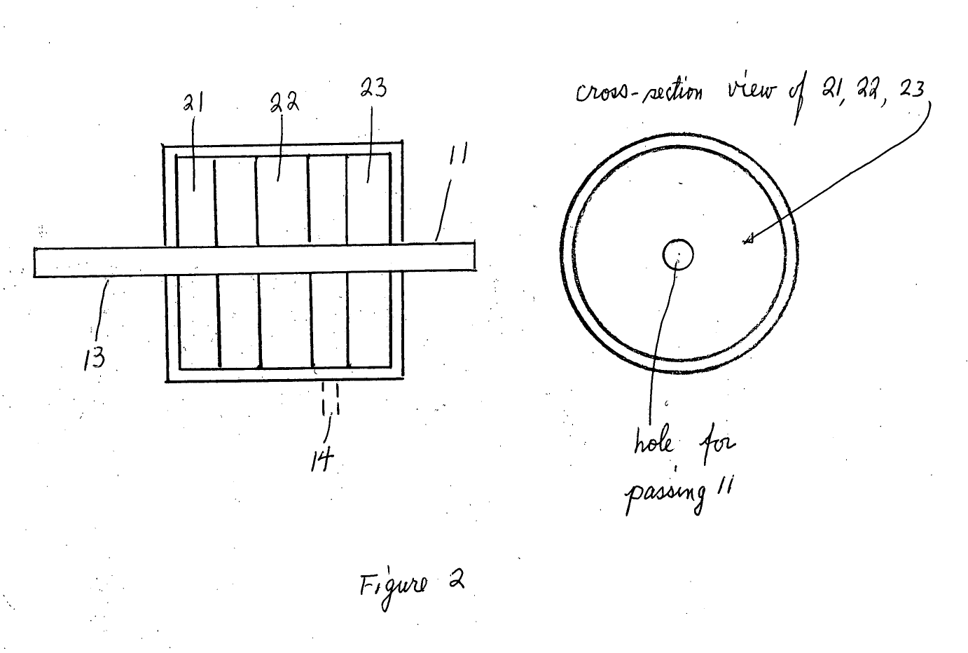

FIG. 2 illustrates in detail the sealing bearing 9 of FIG. 1. There are three identical Sealing sections 21, 22, and 23 which are located as shown in FIG. 2. The pumped fluid which leaks through sealing sections 21 and 22 are returned to the inlet through 14. The sealing section 23 keeps the pumped fluid from leaking out.

In my preferred embodiment, the components 1, 2, 21, 22, and 23 are made of Teflon, or some other material having zero surface affinity with blood.

Having now fully described my invention, what I claim as new and desire to secure by letters patent is:

1. An implantable ventricular assist device comprising a container, inlet and outlet ports in said container, an outer and an inner helical pumping element in said container, the outer element having one more thread than the inner element, the outer element being mounted with bearing(s) for rotation about a first fixed axis, the inner element being mounted with bearing(s) for rotation about a second fixed axis parallel to the said first axis but offset therefrom, and means for driving the said inner element by way of a sealing bearing, whereby the blood being pumped is moved axially in a straight line through the said ventricular assist device. Material having zero surface affinity with blood is used in the sealing and\or mounting bearing.

2. An implantable ventricular assist device according to claim 1, in which the said sealing bearing is located at the outlet end.

3. An implantable ventricular assist device according to claim 2, in which the said sealing bearing has a multiple number N of sealing sections, thereby forming (N-1) separate compartments in the said sealing bearing.

4. An implantable ventricular assist device according to claim 3, in which at least one of the compartments is connected by a conduit to the inlet end.

5. An implantable ventricular assist device according to claim 2 having a tubing shell of the outer element, and attachments to the tubing shell to form vertical bearing surfaces for restricting the movement of the outer element along the axial direction.

6. An implantable ventricular assist device according to claim 5 with an integrated bearing holding the outer element for both rotation and axial movement at each end.

7. An implantable ventricular assist device in which the integrated bearing at the inlet end is formed in such a way that it can also be used as an inlet port.

8. An implantable ventricular assist device according to claim 2 in which the inner element shaft at the inlet end is supported by a spider mounted bearing.
